(19) United States
(12) Patent Application Publication
(10) Pub. No.: US 2014/0065621 A1
(43) Pub. Date: Mar. 6, 2014

(54) METHODS FOR INCREASING FETAL FRACTION IN MATERNAL BLOOD

(71) Applicants: Ravi Mhatre, Menlo Park, CA (US); Johan Baner, San Fracisco, CA (US); Bernhard Zimmermann, San Mateo, CA (US); Matthew Hill, Menlo Park, CA (US); Zachary Demko, Los Altos Hills, CA (US)

(72) Inventors: Ravi Mhatre, Menlo Park, CA (US); Johan Baner, San Fracisco, CA (US); Bernhard Zimmermann, San Mateo, CA (US); Matthew Hill, Menlo Park, CA (US); Zachary Demko, Los Altos Hills, CA (US)

(73) Assignee: Natera, Inc., San Carlos, CA (US)

(21) Appl. No.: 13/793,316

(22) Filed: Mar. 11, 2013

Related U.S. Application Data
(60) Provisional application No. 61/743,423, filed on Sep. 4, 2012.

(51) Int. Cl. C12Q 1/68

(52) U.S. Cl. CPC C12Q 1/6883
USPC 435/6.12

(57) ABSTRACT

The invention provides methods of increasing the fetal fraction in maternal blood and plasma. This increase in fetal fraction improves the accuracy and decreases the “no call” rate for prenatal testing that measures fetal DNA in maternal blood.

### CROSS-REFERENCE TO RELATED APPLICATIONS

This application claims the benefit of U.S. Provisional Application Ser. No. 61/743,423, filed Sep. 4, 2012, which is hereby incorporated by reference in its entirety for the teachings therein.

### FIELD OF THE INVENTION

The present invention generally relates to methods of increasing the fetal fraction in maternal blood and plasma.

### BACKGROUND OF THE INVENTION

There is a great need for methods for non-invasive prenatal testing (NIPT). Non-invasive prenatal testing can be used to test for many conditions; for example, it can be used to determine paternity of a gestating fetus, to determine whether or not a fetus has any whole chromosomal abnormalities such as Down syndrome, Edwards syndrome, or Turner Syndrome, to determine whether or not a fetus has any partial chromosomal abnormalities such as mosaicism, deletion syndromes, or duplications, or to determine the genotype of the fetus at one or a plurality of loci, for example disease linked single nucleotide polymorphisms (SNPs) or short tandem repeats (STRs).

Non-invasive prenatal testing is typically done by measuring fetal DNA that may be present in maternal blood. Recent efforts have focused on isolating fetal cells that may be present in maternal circulation, or on measuring cell free DNA (cfDNA) that is present in the maternal plasma, and which contains a mixture of maternal DNA and fetal DNA. Methods that focus on measuring cfDNA are affected by the fraction of fetal DNA that is present in the maternal plasma. Different methods of measuring the fraction of fetal DNA in the maternal plasma give different results, but typically the percent of DNA that is from the fetus out of the total amount of DNA (fetal fraction, FF) ranges from about 2% to as high as about 40%. The accuracy of the non-invasive prenatal test is typically higher when the fraction of fetal DNA in the maternal plasma is higher. A big challenge for cfDNA based NIPT is that the fetal fraction has a high variance—even at a fixed gestational age the fetal fraction can range by more than an order of magnitude. Samples with high fetal fraction, for example above 10% fetal DNA, typically result in accurate results. However, samples with a low fetal fraction, for example below 5% fetal DNA typically result in very poorly accurate results or a high rate of “no calls” (samples for which a result is not reported due to lack of conclusive data.) Methods that are able to increase the fetal fraction will tend to increase the accuracy of any prenatal tests that rely on measuring fetal cfDNA. One way to conceptualize this is to think of the fetal DNA as the signal, and the maternal DNA as the noise. The more signal present, the easier it is to decipher the signal.

It is believe that the maternal cfDNA in maternal blood originates largely from lysed/apoptotic maternal cells. Likewise, fetal cfDNA in maternal blood is believed to originate from lysed/apoptotic cells whose DNA is fetal in nature. Note that some or all placental cells are typically genetically fetal in nature.

There are a number of methods that have been published that claim to increase fetal fraction in samples ex vivo, that is, after they have been drawn, for example, size exclusion chromatography.

Improved methods are desired for increasing the fetal fraction in maternal blood. Preferably, these methods will require minimal additional steps or additional processing time.

### SUMMARY OF THE INVENTION

In one aspect, the invention features methods for performing non-invasive prenatal testing on a pregnant woman. In some embodiments, the method includes in sequential order (a) administering (i) a nutritious composition (e.g., a food or drink) or (ii) a stimulant to the pregnant woman in an amount sufficient to increase the fetal fraction (i.e., the amount of DNA from fetus divided by the total amount of DNA) in the blood, plasma, or serum of the pregnant woman; (b) obtaining a blood sample from the pregnant woman; and (c) performing non-invasive prenatal testing on the blood sample or a fraction thereof. In some embodiments, the method includes having the pregnant woman consume the nutritious composition (e.g., a food or drink) or the stimulant. In some embodiments, the composition has at least 5, 10, 15, 20, 25, 30, 35, 40, 45, 50, 55, 60, 65, 70, 75, 80, 90, 100, 150, or 200 g carbohydrate. In some embodiments, the composition has at least 0.1, 0.2, 0.3, 0.4, 0.5, 0.6, 0.7, 0.8, 0.9, 1, 1.2, 1.5, 2.0, 2.2, or 2.5 g carbohydrate per kg of body weight. In some embodiments, the food or drink includes fructose. In some embodiments, the composition has at least 50, 75, 100, 150, 175, 190, 200, 250, 300, 350, 400, 450, or 500 calories. In some embodiments, the stimulant includes caffeine, such as at least 20, 25, 30, 35, 40, 45, 50, 55, 60, 65, 70, 75, 80, 90, 100, 150, 200, 250, 300, 350, 400, 450, or 500 mg caffeine.

In one aspect, the invention provides methods for performing non-invasive prenatal testing on a pregnant woman. In some embodiments, the method includes in sequential order (a) having the pregnant woman exercise or palpating or massaging the abdomen of the pregnant woman in an amount sufficient to increase the fetal fraction in the blood, plasma, or serum of the pregnant woman; (b) obtaining a blood sample from the pregnant woman; and (c) performing non-invasive prenatal testing on the blood sample or a fraction thereof.

In some embodiments of any of the aspects of the invention, step (a) increases the fetal fraction in the blood, plasma, or serum of the pregnant woman by at least 10, 15, 20, 25, 30, 35, 40, 45, 50, 55, 60, 65, 70, 75, or 80% (as a percent of the original fetal fraction). In some embodiments, the fetal fraction increases by between 10 to 80%, such as between 15 to 70%, 15 to 54%, 20 to 70%, 20 to 54%, 30 to 70%, 30 to 54%, 30 to 70%, or 30 to 54%, inclusive.

In some embodiments, the time between step (a) and step (b) is between 1 and 180 minutes, such as between 1 to 120 minutes, 1 to 100 minutes, 1 to 60 minutes, 1 to 30 minutes, 5 to 60 minutes, 5 to 30 minutes, 5 to 15 minutes, 10 to 60 minutes, 10 to 30 minutes, or 20 to 60 minutes, inclusive. In some embodiments, the time between step (a) and step (b) is less than 60 minutes, such as less than 45, 30, 25, 20, 15, 10, 5, or 3 minutes.

In some embodiments of any of the aspects of the invention, the prenatal testing determines the presence or absence of a chromosomal abnormality in the genome of the fetus. In some embodiments, the chromosomal abnormality is selected from the group consisting of nullsomy, monosomy, uniparental disomy, trisomy, matched trisomy, unmatched trisomy, maternal trisomy, paternal trisomy, mosaicism tetrasomy, matched tetrasomy, unmatched tetrasomy, other aneuploidies, unbalanced translocations, balanced translocations, insertions, deletions, recombinations, and combinations thereof. In some embodiments, the prenatal testing determines the presence or absence of a euploidy. In some embodiments, the prenatal testing includes determining whether the individual has Down syndrome, Edwards syndrome, Patau syndrome, Klinefelters syndrome, 47,XXX, 47,XYY, Turner syndrome, triploidy, DiGeorge syndrome, Cri du Chat syndrome, Angelman syndrome, Praeder-Willi syndrome, Wolf-Hirschhorn syndrome, Smith-Magenis syndrome, Williams-Beuren syndrome, Phelan-McDermid syndrome, or Sotos Syndrome. In some embodiments, the chromosome of interest is selected from the group consisting of chromosome 13, chromosome 18, chromosome 21, the X chromosome, the Y chromosome, and combinations thereof. In some embodiments, a confidence is computed for the results of the prenatal testing, such as the chromosome copy number determination, disease-linked locus determination, or paternity determination.

In some embodiments of any of the aspects of the invention, the prenatal testing includes measuring genetic material (e.g., measuring using a SNP genotyping array or high throughput DNA sequencing) in the blood sample or fraction thereof and producing genetic data for some or all of the possible alleles at a plurality of loci on the chromosome or chromosome segment of interest; creating on a computer a set of one or more hypotheses about the number of copies of the chromosome or chromosome segment of interest in the genome of the fetus; determining on a computer the probability of each of the hypotheses given the produced genetic data; and using the probabilities associated with each hypothesis to determine the most likely number of copies of the chromosome or chromosome segment of interest in the genome of the fetus.

In some embodiments of any of the aspects of the invention, the prenatal testing includes making genotypic measurements at a plurality of polymorphic loci in the blood sample or fraction thereof; determining on a computer a fetal fraction in the blood sample or fraction thereof given the genotypic measurements of the blood sample or fraction thereof; creating on a computer a set of ploidy state hypotheses for a chromosome or chromosome segment of interest in the fetus; determining on the computer the probability of each of the hypotheses given the genetic measurements of the blood sample or fraction thereof and the fetal fraction; and using the determined probabilities of each hypothesis to determine the most likely copy number of the chromosome or chromosome segment of interest in the genome of the fetus. In some embodiments, the method includes obtaining genotypic measurements at the plurality of polymorphic loci from genetic material from the mother and/or father of the fetus, wherein the probability of each of the hypothesis is determined using the genotypic data of the mother and/or father, the genotypic data of the blood sample or fraction thereof, and the fetal fraction. In some embodiments, the plurality of polymorphic loci comprises (i) a plurality of SNPs from the chromosome of interest and (ii) a plurality of SNPs from at least one chromosome that is expected to be disomic in the fetus. In some embodiments, the method includes obtaining genotypic measurements at each of the SNPs from genetic material from the mother and the father of the fetus; using the genotypic data of the mother and father to determine parental contexts for each of the SNPs; grouping the genotypic measurements of the blood sample or fraction thereof into the parental contexts; using the grouped genotypic measurements from the at least one chromosome that is expected to be disomic to determine a platform response; using the grouped genotypic measurements from the at least one chromosome that is expected to be disomic to determine a fetal fraction in the blood sample or fraction thereof; using the determined platform response and the determined fetal fraction to predict an expected distribution of SNP measurements for each set of SNPs in each parental context under each hypothesis; and calculating the probabilities that each of the hypotheses is true given the platform response, the determined fetal fraction, the grouped genotypic measurements of the blood sample or fraction thereof, and the predicted expected distributions for each parental context, for each hypothesis. In some embodiments, the fetal fraction in the blood sample or fraction thereof is determined for individual chromosomes.

In some embodiments of any of the aspects of the invention, the prenatal testing includes (a) measuring the amount of genetic material on a chromosome or chromosome segment of interest in the blood sample or a fraction thereof; (b) comparing the amount from step (a) to a reference amount; and (c) identifying the presence or absence of a chromosomal abnormality in the genome of the fetus based on the comparison. In some embodiments, the reference amount is (i) a threshold value or (ii) an expected amount for a particular copy number hypothesis. In some embodiments, the reference amount is the amount determined for another chromosome from the same sample that is expected to be disomic. In some embodiments, the reference amount is the amount determined for the same chromosome from one or more different samples. In some embodiments, the reference amount is the mean or median of the values determined for two or more different chromosomes or different samples.

In some embodiments of any of the aspects of the invention, the prenatal testing includes (a) sequencing DNA from the blood sample or fraction thereof to obtain a plurality of sequence tags aligning to target loci; wherein the sequence tags are of sufficient length to be assigned to a specific target locus; wherein the target loci are from a plurality of different chromosomes; and wherein the plurality of different chromosomes comprise at least one first chromosome suspected of having an abnormal distribution in the sample and at least one second chromosome presumed to be normally distributed in the sample; (b) assigning on a computer the plurality of sequence tags to their corresponding target loci; (c) determining on a computer a number of sequence tags aligning to the target loci of the first chromosome and a number of sequence tags aligning to the target loci of the second chromosome; and (d) comparing on a computer the numbers from step (c) to determine the presence or absence of an abnormal distribution of the first chromosome.

In some embodiments of any of the aspects of the invention, the prenatal testing includes amplifying two or more selected polymorphic nucleic acid regions from a first chromosome in the blood sample or fraction thereof; amplifying two or more selected polymorphic nucleic acid regions from a second chromosome; quantifying a relative frequency of each allele from the selected polymorphic nucleic acid regions to determine the fetal fraction in the sample; quantifying a relative frequency of the first and second chromosomes of interest (e.g., a relative frequency based on the selected polymorphic nucleic acid regions or based on selected non-polymorphic nucleic acid regions); and adjusting the relative frequency of the first and second chromosomes of interest based on the fetal fraction to determine the likelihood of a fetal aneuploidy.

In some embodiments of any of the aspects of the invention, the prenatal testing determines the presence or absence of a disease-linked locus in the genome of the fetus. In some embodiments, the locus is linked to a disease selected from the group consisting of cystic fibrosis, Huntington's disease, Fragile X, thallasemia, muscular dystrophy, Alzheimer, Fanconi Anemia, Gaucher Disease, Mucolipidosis IV, Niemann-Pick Disease, Tay-Sachs disease, Sickle cell anemia, Parkinson disease, Torsion Dystonia, and cancer.

In some embodiments of any of the aspects of the invention, the prenatal testing determines whether or not an alleged father is the biological father of the fetus. In some embodiments, the method includes making genotypic measurements at a plurality of polymorphic loci on genetic material from the alleged father; making genotypic measurements at the plurality of polymorphic loci in the blood sample or fraction thereof; determining on a computer the probability that the alleged father is the biological father of the fetus using the genotypic measurements made on the genetic material from the alleged father and the blood sample or fraction thereof; and establishing whether the alleged father is the biological father of the fetus using the determined probability that the alleged father is the biological father of the fetus. In some embodiments, the method also includes obtaining genotypic measurements at the plurality of polymorphic loci from genetic material from the mother, wherein the probability that the alleged father is the biological father of the fetus is determined using the genotypic measurements made on the genetic material from the mother, the genetic material from the alleged father, and the blood sample or fraction thereof.

In one aspect, the invention features a report comprising a result from any of the non-invasive prenatal testing methods of the invention.

In one aspect, the invention features methods of increasing the fetal fraction in the blood of a pregnant woman. In some embodiments, the method includes in sequential order (a) administering (i) a nutritious composition (e.g., a food or drink) or (ii) a stimulant to the pregnant woman in an amount sufficient to increase the increase the fetal fraction in the blood, plasma, or serum of the pregnant woman; (b) obtaining a blood sample from the pregnant woman; and (c) measuring the fetal fraction in the blood sample or a fraction thereof. In some embodiments, the method includes having the pregnant woman consume the nutritious composition (e.g., a food or drink) or the stimulant. In some embodiments, the method further includes performing non-invasive prenatal testing on the blood sample or a fraction thereof.

In some embodiments of any of the aspects of the invention, the method includes determining a fetal fraction in the sample by obtaining genotypic data from the blood sample or fraction thereof for a set of polymorphic loci on at least one chromosome that is expected to be disomic in both the mother and the fetus; creating on a computer a plurality of hypotheses each corresponding to different fetal fractions at the chromosome; building, on a computer, a model for the expected allele measurements in the blood sample or fraction thereof at the set of polymorphic loci on the chromosome for possible fetal fractions; calculating on a computer a relative probability of each of the fetal fraction hypotheses using the model and the allele measurements from the blood sample or fraction thereof; and determining, on a computer, the fetal fraction in the blood sample or fraction thereof by selecting the fetal fraction corresponding to the hypothesis with the greatest probability. In some embodiments, the method also includes obtaining genotypic data for the set of polymorphic loci from the mother of the fetus; optionally obtaining genotypic data for the set of polymorphic loci from the father of the fetus; and determining on a computer the fetal fraction in the blood sample or fraction thereof given the genotypic data of the blood sample or fraction thereof, the genotypic data of mother, and optionally the genotypic data of the father. In some embodiments, the fetal fraction in the blood sample or fraction thereof is determined by identifying those polymorphic loci where the mother is homozygous for a first allele at the polymorphic locus, and the father is (i) heterozygous for the first allele and a second allele or (ii) homozygous for a second allele at the polymorphic locus; and using the amount of the second allele detected in the blood sample or fraction thereof for each of the identified polymorphic loci to determine the fetal fraction in the blood sample or fraction thereof. In some embodiments, the method also includes determining the number of a chromosome of interest in the genome of the fetus using the calculated fetal fraction in the blood sample or fraction thereof. In some embodiments, the method also includes determining the likelihood that the fetal genome contains three copies of a chromosome of interest using the calculated fetal fraction in the blood sample or fraction thereof.

In one aspect, the invention features a method for performing non-invasive prenatal testing on a pregnant woman. In some embodiments, the method involves amplifying DNA from a blood sample or fraction thereof of from the pregnant woman using PCR primers to which a handle has been covalently attached, isolating the amplified DNA by the interaction of the handle with a moiety for which the handle has an affinity, and performing non-invasive prenatal testing on the isolated DNA. In some embodiments of the invention, the handle could be biotin, and the moiety is streptavidin. In some embodiments, the handle and the moiety are complimentary strands of DNA.

In some embodiments of any of the aspects of the invention, the method further comprises performing a clinical action based on the result of the prenatal testing (such as the copy number, disease-linked locus, and/or paternity determination), wherein the clinical action is termination of a pregnancy. In some embodiments, the method further comprises performing amniocentesis or chorion villus biopsy.

In some embodiments of any of the aspects of the invention, the fetal DNA is free floating DNA found in maternal blood or serum. In some embodiments, the fetal DNA is nuclear DNA found in one or more cells from the fetus. In some embodiments, the cell free DNA is measured by sequencing. In some embodiments, the cell free DNA is amplified prior to measurement. In some embodiments, the polymorphic loci are SNPs.

In some embodiments, obtaining genotypic data and/or making genotypic measurements is done by measuring genetic material using techniques selected from the group consisting of padlock probes, circularizing probes, genotyping microarrays, SNP genotyping assays, chip based microarrays, bead based microarrays, other SNP microarrays, other genotyping methods, Sanger DNA sequencing, pyrosequencing, high throughput sequencing, reversible dye terminator sequencing, sequencing by ligation, sequencing by hybridization, other methods of DNA sequencing, other high throughput genotyping platforms, fluorescent in situ hybridization (FISH), comparative genomic hybridization (CGH), array CGH, and combinations thereof. In some embodiments, obtaining genotypic data and/or making genotypic measurements is done on genetic material that is amplified, prior to being measured, using a technique that is selected from the group consisting of Polymerase Chain Reaction (PCR), ligation-mediated PCR, degenerative oligonucleotide primer PCR, Multiple Displacement Amplification (MDA), allele-specific PCR, allele-specific amplification techniques, bridge amplification, padlock probes, circularizing probes, and combinations thereof.

### DEFINITIONS

Single Nucleotide Polymorphism (SNP) refers to a single nucleotide that may differ between the genomes of two members of the same species. The usage of the term should not imply any limit on the frequency with which each variant occurs.

Sequence refers to a DNA sequence or a genetic sequence. It may refer to the primary, physical structure of the DNA molecule or strand in an individual. It may refer to the sequence of nucleotides found in that DNA molecule, or the complementary strand to the DNA molecule. It may refer to the information contained in the DNA molecule as its representation in silico.

Locus refers to a particular region of interest on the DNA of an individual, which may refer to a SNP, the site of a possible insertion or deletion, or the site of some other relevant genetic variation. Disease-linked SNPs may also refer to disease-linked loci.

Polymorphic Allele, also “Polymorphic Locus,” refers to an allele or locus where the genotype varies between individuals within a given species. Some examples of polymorphic alleles include single nucleotide polymorphisms, short tandem repeats, deletions, duplications, and inversions.

Polymorphic Site refers to the specific nucleotides found in a polymorphic region that vary between individuals.

Allele refers to the genes that occupy a particular locus.

Genetic Data also “Genotypic Data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

Genetic Material also “Genetic Sample” refers to physical matter, such as tissue or blood, from one or more individuals comprising DNA or RNA.

Confidence refers to the statistical likelihood that the called SNP, allele, set of alleles, ploidy call, determined number of chromosome segment copies, or paternity determination correctly represents the real genetic state of the individual.

Ploidy Calling, also “Chromosome Copy Number Calling,” or “Copy Number Calling” (CNC), may refer to the act of determining the quantity and/or chromosomal identity of one or more chromosomes present in a cell.

Aneuploidy refers to the state where the wrong number of chromosomes (e.g., the wrong number of full chromosomes or the wrong number of chromosome segments, such as the presence of deletions or duplications of a chromosome segment) is present in a cell. In the case of a somatic human cell it may refer to the case where a cell does not contain 22 pairs of autosomal chromosomes and one pair of sex chromosomes. In the case of a human gamete, it may refer to the case where a cell does not contain one of each of the 23 chromosomes. In the case of a single chromosome type, it may refer to the case where more or less than two homologous but non-identical chromosome copies are present, or where there are two chromosome copies present that originate from the same parent. In some embodiments, the deletion of a chromosome segment is a microdeletion.

Ploidy State refers to the quantity and/or chromosomal identity of one or more chromosomes types in a cell.

Chromosome may refer to a single chromosome copy, meaning a single molecule of DNA of which there are 46 in a normal somatic cell; an example is ‘the maternally derived chromosome 18’. Chromosome may also refer to a chromosome type, of which there are 23 in a normal human somatic cell; an example is ‘chromosome 18’.

Chromosomal Identity may refer to the referent chromosome number, i.e. the chromosome type. Normal humans have 22 types of numbered autosomal chromosome types, and two types of sex chromosomes. It may also refer to the parental origin of the chromosome. It may also refer to a specific chromosome inherited from the parent. It may also refer to other identifying features of a chromosome.

Allelic Data refers to a set of genotypic data concerning a set of one or more alleles. It may refer to the phased, haplotypic data. It may refer to SNP identities, and it may refer to the sequence data of the DNA, including insertions, deletions, repeats and mutations. It may include the parental origin of each allele.

Allelic State refers to the actual state of the genes in a set of one or more alleles. It may refer to the actual state of the genes described by the allelic data.

Allele Count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

Allele Count Probability refers to the number of sequences that are likely to map to a particular locus or a set of alleles at a polymorphic locus, combined with the probability of the mapping. Note that allele counts are equivalent to allele count probabilities where the probability of the mapping for each counted sequence is binary (zero or one). In some embodiments, the allele count probabilities may be binary. In some embodiments, the allele count probabilities may be set to be equal to the DNA measurements.

Allelic Distribution, or “allele count distribution” refers to the relative amount of each allele that is present for each locus in a set of loci. An allelic distribution can refer to an individual, to a sample, or to a set of measurements made on a sample. In the context of digital allele measurements such as sequencing, the allelic distribution refers to the number or probable number of reads that map to a particular allele for each allele in a set of polymorphic loci. In the context of analog allele measurements such as SNP arrays, the allelic distribution refers to allele intensities and/or allele ratios. The allele measurements may be treated probabilistically, that is, the likelihood that a given allele is present for a give sequence read is a fraction between 0 and 1, or they may be treated in a binary fashion, that is, any given read is considered to be exactly zero or one copies of a particular allele.

Allelic Distribution Pattern refers to a set of different allele distributions for different parental contexts. Certain allelic distribution patterns may be indicative of certain ploidy states.

Primer, also “PCR probe” refers to a single DNA molecule (a DNA oligomer) or a collection of DNA molecules (DNA oligomers) where the DNA molecules are identical, or nearly so, and wherein the primer contains a region that is designed to hybridize to a targeted locus (e.g., a targeted polymorphic locus or a non-polymorphic locus) or to a universal priming sequence, and may contain a priming sequence designed to allow PCR amplification. A primer may also contain a molecular barcode. A primer may contain a random region that differs for each individual molecule.

Hybrid Capture Probe refers to any nucleic acid sequence, possibly modified, that is generated by various methods such as PCR or direct synthesis and intended to be complementary to one strand of a specific target DNA sequence in a sample. The exogenous hybrid capture probes may be added to a prepared sample and hybridized through a denature-reannealing process to form duplexes of exogenous-endogenous fragments. These duplexes may then be physically separated from the sample by various means.

Sequence Read refers to data representing a sequence of nucleotide bases that were measured using a clonal sequencing method. Clonal sequencing may produce sequence data representing single, or clones, or clusters of one original DNA molecule. A sequence read may also have associated quality score at each base position of the sequence indicating the probability that nucleotide has been called correctly.

Mapping a sequence read is the process of determining a sequence read's location of origin in the genome sequence of a particular organism. The location of origin of sequence reads is based on similarity of nucleotide sequence of the read and the genome sequence.

Matched Copy Error, also “Matching Chromosome Aneuploidy” (MCA), refers to a state of aneuploidy where one cell contains two identical or nearly identical chromosomes. This type of aneuploidy may arise during the formation of the gametes in meiosis, and may be referred to as a meiotic non-disjunction error. This type of error may arise in mitosis. Matching trisomy may refer to the case where three copies of a given chromosome are present in an individual and two of the copies are identical.

Unmatched Copy Error, also “Unique Chromosome Aneuploidy” (UCA), refers to a state of aneuploidy where one cell contains two chromosomes that are from the same parent, and that may be homologous but not identical. This type of aneuploidy may arise during meiosis, and may be referred to as a meiotic error. Unmatching trisomy may refer to the case where three copies of a given chromosome are present in an individual and two of the copies are from the same parent, and are homologous, but are not identical. Note that unmatching trisomy may refer to the case where two homologous chromosomes from one parent are present, and where some segments of the chromosomes are identical while other segments are merely homologous.

Homologous Chromosomes refers to chromosome copies that contain the same set of genes that normally pair up during meiosis.

Identical Chromosomes refers to chromosome copies that contain the same set of genes, and for each gene they have the same set of alleles that are identical, or nearly identical.

Allele Drop Out (ADO) refers to the situation where at least one of the base pairs in a set of base pairs from homologous chromosomes at a given allele is not detected.

Locus Drop Out (LDO) refers to the situation where both base pairs in a set of base pairs from homologous chromosomes at a given allele are not detected.

Homozygous refers to having similar alleles as corresponding chromosomal loci.

Heterozygous refers to having dissimilar alleles as corresponding chromosomal loci.

Heterozygosity Rate refers to the rate of individuals in the population having heterozygous alleles at a given locus. The heterozygosity rate may also refer to the expected or measured ratio of alleles, at a given locus in an individual, or a sample of DNA.

Chromosomal Region refers to a segment of a chromosome, or a full chromosome.

Segment of a Chromosome refers to a section of a chromosome that can range in size from one base pair to the entire chromosome.

Chromosome refers to either a full chromosome, or a segment or section of a chromosome.

Copies refers to the number of copies of a chromosome segment. It may refer to identical copies, or to non-identical, homologous copies of a chromosome segment wherein the different copies of the chromosome segment contain a substantially similar set of loci, and where one or more of the alleles are different. Note that in some cases of aneuploidy, such as the M2 copy error, it is possible to have some copies of the given chromosome segment that are identical as well as some copies of the same chromosome segment that are not identical.

Haplotype refers to a combination of alleles at multiple loci that are typically inherited together on the same chromosome. Haplotype may refer to as few as two loci or to an entire chromosome depending on the number of recombination events that have occurred between a given set of loci. Haplotype can also refer to a set of SNPs on a single chromatid that are statistically associated.

Haplotypic Data, also “Phased Data” or “Ordered Genetic Data,” refers to data from a single chromosome in a diploid or polyploid genome, i.e., either the segregated maternal or paternal copy of a chromosome in a diploid genome.

Phasing refers to the act of determining the haplotypic genetic data of an individual given unordered, diploid (or polyploidy) genetic data. It may refer to the act of determining which of two genes at an allele, for a set of alleles found on one chromosome, are associated with each of the two homologous chromosomes in an individual.

Phased Data refers to genetic data where one or more haplotypes have been determined.

Hypothesis refers to a possible ploidy state at a given set of one or more chromosomes, a possible allelic state at a given set of one or more loci, a possible paternity relationship, or a possible fetal fraction at a given set of one or more chromosomes. The set of possibilities may comprise one or more elements.

Copy Number Hypothesis, also “Ploidy State Hypothesis,” refers to a hypothesis concerning the number of copies of a chromosome in an individual. It may also refer to a hypothesis concerning the identity of each of the chromosomes, including the parent of origin of each chromosome, and which of the parent's two chromosomes are present in the individual. It may also refer to a hypothesis concerning which chromosomes, or chromosome segments, if any, from a related individual correspond genetically to a given chromosome from an individual.

Related Individual refers to any individual who is genetically related to, and thus shares haplotype blocks with, the target individual. In one context, the related individual may be a genetic parent of the target individual, or any genetic material derived from a parent, such as a sperm, a polar body, an embryo, a fetus, or a child. It may also refer to a sibling, parent or a grandparent.

Sibling refers to any individual whose genetic parents are the same as the individual in question. In some embodiments, it may refer to a born child, an embryo, or a fetus, or one or more cells originating from a born child, an embryo, or a fetus. A sibling may also refer to a haploid individual that originates from one of the parents, such as a sperm, a polar body, or any other set of haplotypic genetic matter. An individual may be considered to be a sibling of itself.

DNA of Fetal Origin refers to DNA that was originally part of a cell whose genotype was essentially equivalent to that of the fetus.

DNA of Maternal Origin refers to DNA that was originally part of a cell whose genotype was essentially equivalent to that of the mother.

Child may refer to an embryo, a blastomere, or a fetus. Note that in the presently disclosed embodiments, the concepts described apply equally well to individuals who are a born child, a fetus, an embryo or a set of cells therefrom. The use of the term child may simply be meant to connote that the individual referred to as the child is the genetic offspring of the parents.

Parent refers to the genetic mother or father of an individual. An individual typically has two parents, a mother and a father, though this may not necessarily be the case such as in genetic or chromosomal chimerism. A parent may be considered to be an individual.

Parental Context refers to the genetic state of a given SNP, on each of the two relevant chromosomes for one or both of the two parents of the target.

Maternal Plasma refers to the plasma portion of the blood from a female who is pregnant.

Clinical Decision refers to any decision to take or not take an action that has an outcome that affects the health or survival of an individual. In the context of prenatal diagnosis, a clinical decision may refer to a decision to abort or not abort a fetus. A clinical decision may also refer to a decision to conduct further testing, to take actions to mitigate an undesirable phenotype, or to take actions to prepare for the birth of a child with abnormalities.

Diagnostic Box refers to one or a combination of machines designed to perform one or a plurality of aspects of the methods disclosed herein. In an embodiment, the diagnostic box may be placed at a point of patient care. In an embodiment, the diagnostic box may perform targeted amplification followed by sequencing. In an embodiment the diagnostic box may function alone or with the help of a technician.

Informatics Based Method refers to a method that relies heavily on statistics to make sense of a large amount of data. In the context of prenatal diagnosis, it refers to a method designed to determine the ploidy state at one or more chromosomes, the allelic state at one or more alleles, or paternity by statistically inferring the most likely state, rather than by directly physically measuring the state, given a large amount of genetic data, for example from a molecular array or sequencing. In an embodiment of the present disclosure, the informatics based technique may be one disclosed in this patent application. In an embodiment of the present disclosure it may be PARENTAL SUPPORT.

Primary Genetic Data refers to the analog intensity signals that are output by a genotyping platform. In the context of SNP arrays, primary genetic data refers to the intensity signals before any genotype calling has been done. In the context of sequencing, primary genetic data refers to the analog measurements, analogous to the chromatogram, that comes off the sequencer before the identity of any base pairs have been determined, and before the sequence has been mapped to the genome.

Secondary Genetic Data refers to processed genetic data that are output by a genotyping platform. In the context of a SNP array, the secondary genetic data refers to the allele calls made by software associated with the SNP array reader, wherein the software has made a call whether a given allele is present or not present in the sample. In the context of sequencing, the secondary genetic data refers to the base pair identities of the sequences have been determined, and possibly also where the sequences have been mapped to the genome.

Preferential Enrichment of DNA that corresponds to a locus, or preferential enrichment of DNA at a locus, refers to any method that results in the percentage of molecules of DNA in a post-enrichment DNA mixture that correspond to the locus being higher than the percentage of molecules of DNA in the pre-enrichment DNA mixture that correspond to the locus. The method may involve selective amplification of DNA molecules that correspond to a locus. The method may involve removing DNA molecules that do not correspond to the locus. The method may involve a combination of methods. The degree of enrichment is defined as the percentage of molecules of DNA in the post-enrichment mixture that correspond to the locus divided by the percentage of molecules of DNA in the pre-enrichment mixture that correspond to the locus. Preferential enrichment may be carried out at a plurality of loci. In some embodiments of the present disclosure, the degree of enrichment is greater than 20, 200, or 2,000. When preferential enrichment is carried out at a plurality of loci, the degree of enrichment may refer to the average degree of enrichment of all of the loci in the set of loci.

Amplification refers to a method that increases the number of copies of a molecule of DNA.

Selective Amplification may refer to a method that increases the number of copies of a particular molecule of DNA, or molecules of DNA that correspond to a particular region of DNA. It may also refer to a method that increases the number of copies of a particular targeted molecule of DNA, or targeted region of DNA more than it increases non-targeted molecules or regions of DNA. Selective amplification may be a method of preferential enrichment.

Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences.

Universal Adapters, or “ligation adaptors” or “library tags” are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

Targeting refers to a method used to selectively amplify or otherwise preferentially enrich those molecules of DNA that correspond to a set of loci in a mixture of DNA.

Joint Distribution Model refers to a model that defines the probability of events defined in terms of multiple random variables, given a plurality of random variables defined on the same probability space, where the probabilities of the variable are linked. In some embodiments, the degenerate case where the probabilities of the variables are not linked may be used.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 is a graph of the fetal fraction (%) for nine women where the fetal fraction measured before drinking orange juice is plotted against the fetal fraction measured after drinking orange juice. The dots above the x=y line indicate cases where the fetal fraction increased after drinking the orange juice (8/9) and the dot below the x=y line indicates the case where the fetal fraction decreased after drinking the orange juice.

FIG. 2 is a graph of the fetal fraction for 13 women, as described for FIG. 1.

FIG. 3 is a graph of the fetal fraction for 22 women, as described for FIG. 1.

### DETAILED DESCRIPTION OF THE INVENTION

Presented here are a number of ex vivo and in vivo methods for increasing the fetal fraction in maternal blood, so as to result in an increase in the fetal fraction of the maternal plasma after it is isolated, and thus result in an increase in test accuracy for those samples drawn from women after the methods have been performed. As described further below, the consumption of a carbohydrate-containing drink increased the fetal fraction in maternal blood from pregnant women at various time points in their first, second, or third trimester. Surprisingly, the increase in fetal fraction was greater for fetuses in the first trimester than in the second trimester even though fetuses were previously thought to be less effected by the consumption of carbohydrates or other nutritious substances in the first trimester than in the second trimester. These methods may be used to increase the accuracy and to decrease the “no call” rate for prenatal testing of fetal DNA from maternal blood, such as fetal aneuploidy screening, screening for the inheritance of a disease-linked locus, or paternity testing.

### In Vivo Methods

In one embodiment, the method for increasing fetal fraction in maternal blood may comprise the pregnant woman consuming a specific foodstuff (such as any of the compositions, foods, drinks, or stimulants described herein) prior to drawing blood. In one embodiment, the foodstuff may be a drink or food that is high in sugar (such as fructose or glucose) or some stimulant. In one embodiment the method comprises a pregnant woman consuming a sugar and/or carbohydrate and/or calorie rich food or beverage, drawing blood, and performing NIPT on the blood sample. In one embodiment the method comprises a woman drinking a beverage containing sugar, drawing blood from the pregnant woman, and performing NIPT on the blood sample. In one embodiment the method comprises a pregnant woman consuming a drink or food containing caffeine or other stimulant, drawing blood from the pregnant woman, and performing NIPT on the blood sample. In one embodiment the method includes administering a composition (e.g., a composition that includes a nutritious substance such as a carbohydrate, caloric substance, or stimulant) to the pregnant woman, drawing blood from the pregnant woman, and performing NIPT on the blood sample. In some embodiments, the time between administering the composition and obtaining the blood sample is between 1 and 180 minutes, such as between 1 to 120 minutes, 1 to 100 minutes, 1 to 60 minutes, 1 to 30 minutes, 5 to 60 minutes, 5 to 30 minutes, 5 to 15 minutes, 10 to 60 minutes, 10 to 30 minutes, or 20 to 60 minutes, inclusive. In some embodiments, the time between these steps is less than 60 minutes, such as less than 45, 30, 25, 20, 15, 10, 5, or 3 minutes. In some embodiments, the method increases the fetal fraction in the blood, plasma, or serum of the pregnant woman by at least 10, 15, 20, 25, 30, 35, 40, 45, 50, 55, 60, 65, 70, 75, or 80%. In some embodiments, the fetal fraction increases by between 10 to 80%, such as between 15 to 70%, 15 to 54%, 20 to 70%, 20 to 54%, 30 to 70%, 30 to 54%, 30 to 70%, or 30 to 54%, inclusive.

Without being held to any particular explanation, one possible explanation for the efficacy of this method is that a rise in blood sugar may result in increased fetal movement which may increase apoptosis and/or lysis of cells proximal to the fetus while the resultant increase in maternal movement may not cause as significant levels of apoptosis of maternal cells. Consumption of sugar rich beverages has been used in the past to increase the movement of the fetus so that better ultrasounds could be performed. It was not obvious that increased fetal movement would result in a higher fetal fraction, especially during the first trimester when fetal movement is minimal.

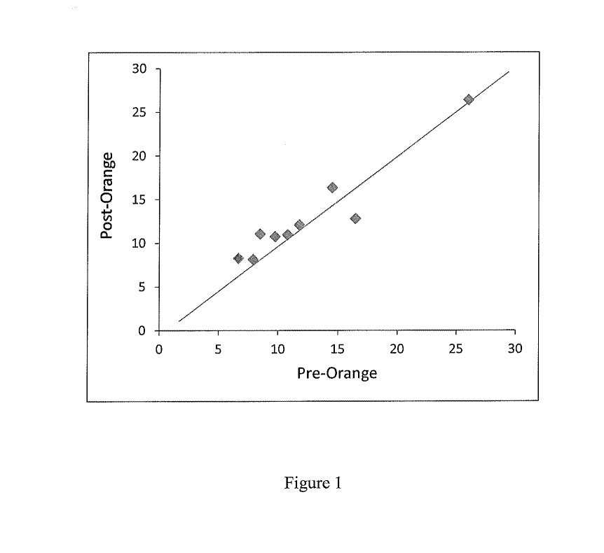

In an experiment involving nine pregnant women, the data found a seven percent increase in fetal fraction after the women drank orange juice. The data is shown in Table 1 and FIG. 1. A seven percent increase in fetal fraction would result in a significant increase in the accuracy of NIPT based on fetal cfDNA.

In the experiment described further in Example 1, a phlebotomist drew two tubes of blood from the nine pregnant women. Each of the nine women then drank 13.5 oz Simply Orange Juice, which, according to the nutrition label, has 45 g carbohydrates. The women waited twenty minutes, and the phlebotomists then drew another two tubes of blood.

TABLE 1
Fetal fraction (%) for nine women as measured on samples drawn
before (pre-OJ) and after (post-OJ) drinking orange juice.

| Patient ID | Pre-OJ FF | Post-OJ FF | Post-OJ FF/Pre-OJ FF |
|---|---|---|---|
| 8392 | 8.51 | 11.04 | 1.30 |
| 8507 | 14.53 | 16.33 | 1.12 |
| 9249 | 6.68 | 8.26 | 1.24 |
| 9282 | 10.79 | 10.94 | 1.01 |
| 9361 | 7.93 | 8.12 | 1.02 |
| 9742 | 26.01 | 26.39 | 1.01 |
| 10241 | 16.47 | 12.79 | 0.78 |
| 10246 | 9.75 | 10.75 | 1.10 |
| 12755 | 11.77 | 12.07 | 1.03 |
| Average | 12.49 | 12.97 | 1.07 |

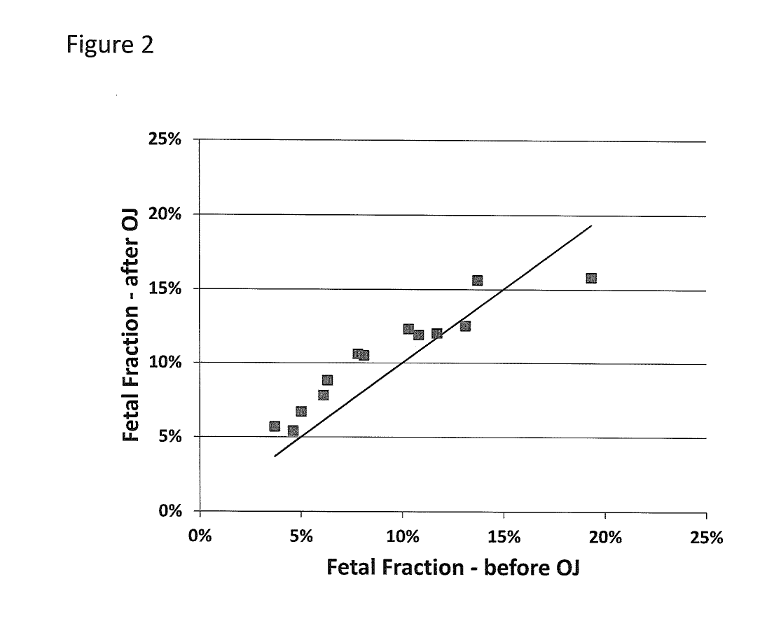

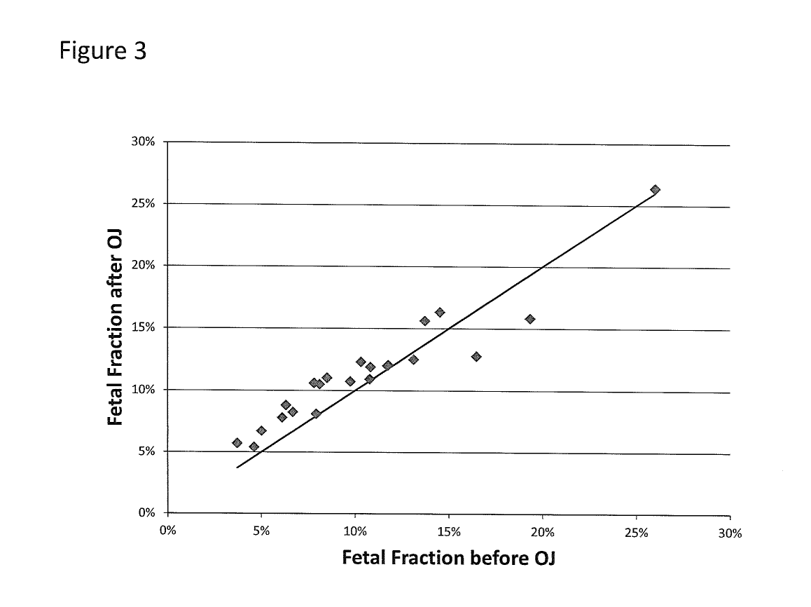

In addition to the nine woman tested above, this experiment was performed for another 13 women. Overall, the 22 woman had an average gestational age of 17 weeks, 5 days (FIG. 3, Table 2). For all 22 women, the average increase in fetal fraction after drinking orange juice was 14.7% (FIG. 3, Table 2). For the 13 women with GA prior to 18 weeks, the average GA was 13 weeks, 2 days, and the average increase in fetal fraction after drinking orange juice was 20.1% (FIG. 2). The p-value is 0.019, comparing to the case where no nutritive substance was consumed and fetal fraction levels are assumed to be on average unchanged, indicating that this is a significant finding.

TABLE 2
Fetal fraction (%) for 22 women as measured on samples drawn
before (pre-OJ) and after (post-OJ) drinking orange juice.

| | | | | Gestational |
|---|---|---|---|---|
| Patient ID | Pre-OJ FF | Post-OJ FF | Increase | Age (GA) |
| 8392 | 8.51% | 11.04% | 30% | 198 |
| 8507 | 14.53% | 16.33% | 12% | 185 |
| 9249 | 6.68% | 8.26% | 24% | 177 |
| 9282 | 10.79% | 10.94% | 1% | 147 |
| 9361 | 7.93% | 8.12% | 2% | 182 |
| 9742 | 26.01% | 26.39% | 1% | 161 |
| 10241 | 16.47% | 12.79% | −22% | 164 |
| 10246 | 9.75% | 10.75% | 10% | 173 |
| 12755 | 11.77% | 12.07% | 3% | 139 |
| 15868 | 5.01% | 6.71% | 34% | 94 |
| 15964 | 10.82% | 11.92% | 10% | 99 |
| 16150 | 7.82% | 10.62% | 36% | 93 |
| 16399 | 4.61% | 5.41% | 17% | 93 |
| 17638 | 8.12% | 10.52% | 30% | 93 |
| 17916 | 13.13% | 12.53% | −5% | 80 |
| 18034 | 3.71% | 5.71% | 54% | 126 |
| 18269 | 11.72% | 12.02% | 3% | 89 |
| 18379 | 6.11% | 7.82% | 28% | 91 |
| 18689 | 13.73% | 15.63% | 14% | 104 |
| 18693 | 10.32% | 12.32% | 19% | 88 |
| 18850 | 19.34% | 15.83% | −18% | 104 |
| 21147 | 6.31% | 8.82% | 40% | 52 |

Exemplary compositions that can be consumed by or otherwise administered to the pregnant woman include compositions that include a nutritious substance (e.g., a carbohydrate), caloric substance, or stimulant. In some embodiments, a nutritious food or drink or a stimulant is consumed by or otherwise administered to the pregnant woman. Exemplary foods or drinks include one or more carbohydrates (e.g., fructose or glucose) and/or stimulants. In some embodiments, the food or drink is a fruit or fruit juice (such as orange, grape, and/or apple juice). In some embodiments, the food or drink provides calories. A composition may have one or more active compounds. In some embodiments, the composition has one or more inactive compounds, such as a stabilizer or filler.

In some embodiments, the composition has at least 5, 10, 15, 20, 25, 30, 35, 40, 45, 50, 55, 60, 65, 70, 75, 80, 90, 100, 150, or 200 g carbohydrate (such as fructose). In some embodiments, the composition includes between 10 to 200 g carbohydrate, such as between 20 to 200 g carbohydrate, 20 to 100 g carbohydrate, 20 to 50 g carbohydrate, 30 to 200 g carbohydrate, 30 to 100 g carbohydrate, 30 to 50 g carbohydrate, 40 to 200 g carbohydrate, 40 to 100 g carbohydrate, or 40 to 60 g carbohydrate, inclusive. In some embodiments, the composition has at least 0.1, 0.2, 0.3, 0.4, 0.5, 0.6, 0.7, 0.8, 0.9, 1, 1.2, 1.5, 2.0, 2.2, or 2.5 g carbohydrate per kg of body weight. In some embodiments, the composition includes between 0.1 to 2.5 g carbohydrate per kg of body weight, such as between 0.2 to 2 g/kg, 0.2 to 1 g/kg, 0.4 to 2 g/kg, 0.4 to 1 g/kg, 0.4 to 0.8 g/kg, 0.6 to 2 g/kg, 0.6 to 1 g/kg, or 0.6 to 0.8 g/kg, inclusive.

In some embodiments, the composition has at least 50, 75, 100, 150, 175, 190, 200, 250, 300, 350, 400, 450, or 500 calories. In some embodiments, the composition includes between 50 to 500 calories, such as between 50 to 400 calories, 50 to 200 calories, 100 to 400 calories, or 100 to 200 calories, inclusive.

In some embodiments, the stimulant includes caffeine, such as at least 20, 25, 30, 35, 40, 45, 50, 55, 60, 65, 70, 75, 80, 90, 100, 150, 200, 250, 300, 350, 400, 450, or 500 mg caffeine. In some embodiments, the amount of caffeine is between 20 to 500 mg, such as between 25 to 400 mg, 25 to 200 mg, 50 to 400 mg, 50 to 200 mg, 50 to 100 mg, or 100 to 200 mg, inclusive.

To facilitate administration of the composition, it may be administered in the form of a food or drink that is consumed by the pregnant woman. Alternatively or additionally, the compositions can be formulated and administered to the pregnant woman using any other method known to those of skill in the art (see, e.g., U.S. Pat. Nos. 8,389,578 and 8,389,557, which are each hereby incorporated by reference in its entirety). General techniques for formulation and administration are found in “Remington: The Science and Practice of Pharmacy,” 21st Edition, Ed. David Troy, 2006, Lippincott Williams & Wilkins, Philadelphia, Pa., which is hereby incorporated by reference in its entirety). Liquids, slurries, tablets, capsules, pills, powders, granules, gels, ointments, suppositories, injections, inhalants, and aerosols are examples of such formulations. By way of example, modified or extended release oral formulation can be prepared using additional methods known in the art. For example, a suitable extended release form of an active ingredient may be a matrix tablet or capsule composition. Suitable matrix forming materials include, for example, waxes (e.g., carnauba, bees wax, paraffin wax, ceresine, shellac wax, fatty acids, and fatty alcohols), oils, hardened oils or fats (e.g., hardened rapeseed oil, castor oil, beef tallow, palm oil, and soya bean oil), and polymers (e.g., hydroxypropyl cellulose, polyvinylpyrrolidone, hydroxypropyl methyl cellulose, and polyethylene glycol). Other suitable matrix tabletting materials are microcrystalline cellulose, powdered cellulose, hydroxypropyl cellulose, ethyl cellulose, with other carriers, and fillers. Tablets may also contain granulates, coated powders, or pellets. Tablets may also be multi-layered. Optionally, the finished tablet may be coated or uncoated.

Typical routes of administering such compositions include, without limitation, oral, sublingual, buccal, topical, transdermal, inhalation, parenteral (e.g., intravenous, intramuscular, intrasternal injection, or infusion techniques), rectal, vaginal, and intranasal. Compositions of the invention are formulated so as to allow the active ingredient(s) contained therein to be bioavailable upon administration of the composition to a pregnant woman. Compositions may take the form of one or more dosage units.

### Additional Methods

Without being bound to any particular mechanism, any method that results in a greater proportion of fetal and/or placental cells than maternal cells apoptosing or otherwise lysing and releasing their DNA into the blood will tend to have the effect of increasing the fetal fraction in the maternal blood. In one embodiment the method comprises a pregnant woman performing an exercise (such as a vigorous exercise), drawing blood from the woman, and performing NIPT on the blood sample.

In one embodiment the method comprises a pregnant woman performing sit ups, crunches or other motion involving abdominal contractions, drawing blood, and performing NIPT on the blood sample. In one embodiment the method comprises performing massage, rubbing or otherwise flexing the abdominal region of a pregnant woman, drawing blood from the woman, and performing NIPT on the blood sample.

In some embodiments, the time between exercising or manipulating the abdomen and obtaining the blood sample is between 1 and 180 minutes, such as between 1 to 120 minutes, 1 to 100 minutes, 1 to 60 minutes, 1 to 30 minutes, 5 to 60 minutes, 5 to 30 minutes, 5 to 15 minutes, 10 to 60 minutes, 10 to 30 minutes, or 20 to 60 minutes, inclusive. In some embodiments, the time between these steps is less than 60 minutes, such as less than 45, 30, 25, 20, 15, 10, 5, or 3 minutes. In some embodiments, the method increases the fetal fraction in the blood, plasma, or serum of the pregnant woman by at least 10, 15, 20, 25, 30, 35, 40, 45, 50, 55, 60, 65, 70, 75, or 80%. In some embodiments, the fetal fraction increases by between 10 to 80%, such as between 15 to 70%, 15 to 54%, 20 to 70%, 20 to 54%, 30 to 70%, 30 to 54%, 30 to 70%, or 30 to 54%, inclusive.

### Measuring Fetal Fraction

If desired, the fetal fraction can be measured before and/or after the administration of a composition, performance of an exercise, and/or manipulation of the abdomen of a pregnant woman using the methods described herein or any known method for measuring fetal fraction. An exemplary method is described in Example 1 that includes amplifying the DNA from a blood sample, sequencing the amplicons, and analyzing the sequencing data using the PARENTAL SUPPORT™ methodology.

In some embodiments, the fetal fraction is determined by obtaining genotypic data from the blood sample (or fraction thereof) for a set of polymorphic loci on at least one chromosome that is expected to be disomic in both the mother and the fetus; creating a plurality of hypotheses each corresponding to different possible fetal fractions at the chromosome; building a model for the expected allele measurements in the blood sample at the set of polymorphic loci on the chromosome for possible fetal fractions; calculating a relative probability of each of the fetal fractions hypotheses using the model and the allele measurements from the blood sample or fraction thereof; and determining the fetal fraction in the blood sample by selecting the fetal fraction corresponding to the hypothesis with the greatest probability. In some embodiments, the method also includes obtaining genotypic data for the set of polymorphic loci from the mother of the fetus; optionally obtaining genotypic data for the set of polymorphic loci from the father of the fetus; and determining, the fetal fraction given the genotypic data of the blood sample, the genotypic data of mother, and optionally the genotypic data of the father. In some embodiments, the fetal fraction is determined by identifying those polymorphic loci where the mother is homozygous for a first allele at the polymorphic locus, and the father is (i) heterozygous for the first allele and a second allele or (ii) homozygous for a second allele at the polymorphic locus; and using the amount of the second allele detected in the blood sample for each of the identified polymorphic loci to determine the fetal fraction in the blood sample.

In some embodiments, the method further includes performing non-invasive prenatal testing on the blood sample (or a fraction thereof).

### Prenatal Testing

A blood sample (or fraction thereof) from a pregnant woman who has undergone any of the methods of the invention to increase the fetal fraction can be used in any of the prenatal testing methods described herein or in any other known prenatal testing methods. Exemplary prenatal methods include those that test for fetal chromosome abnormalities, the inheritance of a disease-linked locus, and/or paternity. In some embodiments, a clinical action is performed based on the results of the prenatal testing. In some embodiments, the clinical action is termination or maintenance of the pregnancy. In some embodiments, the clinical action includes performing additional testing on the fetus, such as amniocentesis or chorion villus biopsy.

### PARENTAL SUPPORT™ Methodology

For prenatal testing, some embodiments may be used in combination with the PARENTAL SUPPORT™ (PS) method, embodiments of which are described in U.S. application Ser. No. 11/603,406 (US Publication No. 20070184467), U.S. application Ser. No. 12/076,348 (US Publication No. 20080243398), U.S. application Ser. No. 13/110,685 (U.S. Publication No. 2011/0288780), PCT Application PCT/US09/52730 (PCT Publication No. WO/2010/017214), and PCT Application No. PCT/US10/050,824 (PCT Publication No. WO/2011/041485), U.S. application Ser. No. 13/300,235 (U.S. Publication No. 2012/0270212), U.S. application Ser. No. 13/335,043 (U.S. Publication No. 2012/0122701), U.S. application Ser. No. 13/683,604, and U.S. application Ser. No. 13/780,022 which are incorporated herein by reference in their entirety. PARENTAL SUPPORT™ is an informatics based approach that can be used to analyze genetic data. In some embodiments, the methods disclosed herein may be considered as part of the PARENTAL SUPPORT™ method. In some embodiments, The PARENTAL SUPPORT™ method is a collection of methods that may be used to determine the genetic data of a target individual, with high accuracy, of one or a small number of cells from that individual, or of a mixture of DNA consisting of DNA from the target individual and DNA from one or a plurality of other individuals (e.g., related individuals), specifically to determine disease-related alleles, other alleles of interest, genetic relationships between individuals (such as paternity), and/or the ploidy state of one or a plurality of chromosomes in the target individual. PARENTAL SUPPORT™ may refer to any of these methods. PARENTAL SUPPORT™ is an example of an informatics based method.

Assume a generalized example where the possible alleles at one locus are A and B; assignment of the identity A or B to particular alleles is arbitrary. Parental genotypes for a particular SNP, termed genetic contexts, are expressed as maternal genotype|paternal genotype. Thus, if the mother is homozygous and the father is heterozygous, this would be represented as AA|AB. Similarly, if both parents are homozygous for the same allele, the parental genotypes would be represented as AA|AA. Furthermore, the fetus would never have AB or BB states and the number of sequence reads with the B allele will be low, and thus can be used to determine the noise responses of the assay and genotyping platform, including effects such as low level DNA contamination and sequencing errors; these noise responses are useful for modeling expected genetic data profiles. There are only five possible maternal|paternal genetic contexts: AA|AA, AA|AB, AB|AA, AB|AB, and AA|BB; other contexts are equivalent by symmetry. SNPs where the parents are homozygous for the same allele are only informative for determining noise and contamination levels. SNPs where the parents are not homozygous for the same allele are informative in determining fetal fraction and copy number count.

Let N_{A,i} and N_{B,i} represent the number of reads of each allele at SNP i, and let Ci represent the parental genetic context at that locus. The data set for a particular chromosome is represented by N_{AB}={N_{A,i}, N_{B,i}}i=1 . . . N and C={Cᵢ}, i=1 . . . N. For each individual chromosome under study, let H represent the set of hypotheses for the total number of chromosomes, the parental origin of each chromosome, and the positions on the parent chromosomes where recombination occurred during formation of the gametes that fertilized to create the child. In some embodiments, five copy number hypotheses are considered by the algorithm for chromosomes 13, 18, 21, and X were maternal monosomy, paternal monosomy, disomy, maternal trisomy, and paternal trisomy. Presence or absence of Y was considered for the Y chromosome. Other copy number hypotheses, such as uniparental disomy, parental origin of the extra chromosome in a trisomy, or differentiating between one and two copies of Y can be included in the algorithm as desired. The probability of a hypothesis P(H) can be computed using the data from the HapMap database and prior information related to each of the ploidy states. For the sake of simplicity, for this dataset no priors were used, i.e. all ploidy states were considered equally likely and the probability of aneuploidy originating on either parent's chromosome was considered equally probable.

Furthermore, let F represent the fetal cfDNA fraction in the sample. Given a set of possible H, C, and F, one can compute the probability of N_{AB},P(N_{AB}|H,F,C) based on modeling the noise sources of the molecular assay and the sequencing platform. The goal is to find the hypothesis H and the fetal fraction F that maximizes P(H,F|N_{AB}). Using standard Bayesian statistical techniques, and assuming a uniform probability distribution for F from 0 to 1, this can be recast in terms of maximizing the probability of P(N_{AB}|H,F,C)P(H) over H and F, all of which can now be computed. The probability of all hypotheses associated with a particular ploidy state and fetal fraction, e.g., trisomy and F=10%, but covering all possible parental chromosome origins and crossover locations, are summed. The ploidy state hypothesis with the highest probability is selected as the test result, the fetal fraction associated with that hypothesis reveals the fetal fraction, and the probability associated with that hypothesis is the calculated accuracy of the result. The calculated accuracy, formally called a confidence, is specific to each test sample.

Different probability distributions associated with the number of A and B reads over a set of SNPs from the various parental contexts are expected for different fetal copy numbers, and the observed distributions inform the algorithm as to the correct fetal copy number.

The data model and the algorithm itself were constructed previously using a number of sets of data that included mixing experiments, a separate set of triad samples (pregnant woman and biological father), computer simulations, and de novo theoretical calculations.

The algorithm uses in silico simulations to generate a very large number of hypothetical sequencing data sets that could result from the possible fetal genetic inheritance patterns, sample parameters, and amplification and measurement artifacts of the method. More specifically, the algorithm first utilizes parental genotypes at each of 11,000 or more SNPs and crossover frequency data from the HapMap database to predict possible fetal genotypes. It then predicts expected data profiles for the sequencing data that would be measured from plasma samples originating from a mother carrying a fetus with each of the possible fetal genotypes and taking into account a variety of parameters including fetal fraction, expected read depth profile, fetal genome equivalents present in the sample, expected amplification bias at each of the SNPs, and a number of noise parameters. A data model describes how the sequencing data is expected to appear for each of these hypotheses given the particular parameter set.

When the actual measured data resembles one set of ploidy state hypotheses (e.g., trisomy) much more closely than another (e.g., euploidy) then the sample is considered to have a high calculated accuracy. When the actual measured data from a sample similarly resembles more than one set of hypotheses (e.g., both trisomy and euploidy) then the sample is considered to have a low calculated accuracy, and the result is a “no call.’ The actual likelihoods are calculated as part of the statistical calculations. This allows for aneuploidy identification at very low fetal fraction, improves call accuracy, and is how each individual chromosome call is assigned a sample-specific calculated accuracy.

### Aneuploidy Screening Methods

Exemplary chromosomal abnormalities that can be detected using the methods of the invention include nullsomy, monosomy, uniparental disomy, trisomy, matched trisomy, unmatched trisomy, maternal trisomy, paternal trisomy, mosaicism tetrasomy, matched tetrasomy, unmatched tetrasomy, other aneuploidies, unbalanced translocations, balanced translocations, insertions, deletions, recombinations, and combinations thereof. In some embodiments, the prenatal testing determines the presence or absence of a euploidy. In some embodiments, the prenatal testing comprises determining whether the individual has Down syndrome, Edwards syndrome, Patau syndrome, Klinefelters syndrome, 47XXX, 47,XYY, Turner syndrome, triploidy, DiGeorge syndrome, Cri du Chat syndrome, Angelman syndrome, Praeder-Willi syndrome, Wolf-Hirschhorn syndrome, Smith-Magenis syndrome, Williams-Beuren syndrome, Phelan-McDermid syndrome, or Sotos Syndrome. In some embodiments, the chromosome of interest is selected from the group consisting of chromosome 13, chromosome 18, chromosome 21, the X chromosome, the Y chromosome, and combinations thereof. In some embodiments, a confidence is computed for the chromosome copy number determination.

In some embodiments for fetal aneuploidy screening, the genetic material in the blood sample is measured (e.g., measuring using a SNP genotyping array or high throughput DNA sequencing) to produce genetic data for some or all of the possible alleles at a plurality of loci on a chromosome or chromosome segment of interest (see, e.g., U.S. application Ser. No. 13/300,235 (U.S. Publication No. 2012/0270212), U.S. application Ser. No. 13/683,604, and U.S. application Ser. No. 13/780,022, which are each hereby incorporated by reference in its entirety). In some embodiments, a set of one or more hypotheses is created about the number of copies of the chromosome or chromosome segment of interest in the genome of the fetus, and the probability of each of the hypotheses given the produced genetic data is determined. In some embodiments, the most likely number of copies of the chromosome or chromosome segment of interest in the genome of the fetus is determined using the probabilities associated with each hypothesis.

In some embodiments, the fetal aneuploidy screening includes making genotypic measurements at a plurality of polymorphic loci in the blood sample (or fraction thereof), and determining a fetal fraction in the blood sample or fraction thereof given the genotypic measurements of the blood sample. In some embodiments, a set of ploidy state hypotheses for a chromosome or chromosome segment of interest in the fetus is created. In some embodiments, the probability of each of the hypotheses is determined given the genetic measurements of the blood sample and the fetal fraction. In some embodiments, the most likely copy number of the chromosome or chromosome segment of interest in the genome of the fetus is determined using the probabilities of each hypothesis. In some embodiments, the method includes obtaining genotypic measurements at the plurality of polymorphic loci from genetic material from the mother and/or father of the fetus, and the probability of each of the hypothesis is determined using the genotypic data of the mother and/or father, the genotypic data of the blood sample, and the fetal fraction.

In some embodiments, the fetal aneuploidy screening includes comparing the amount of a chromosome or chromosome segment of interest to a reference amount or to the amount of a reference chromosome or chromosome segment (see, e.g. U.S. Publication No. 2007/0184467 and U.S. Pat. No. 7,888,017; 8,008,018; 8,296,076; or 8,195,415, which are each hereby incorporated by reference in its entirety). In some embodiments, random (e.g., massively parallel shotgun sequencing) or targeted sequencing is used to determine the amount of one or more chromosomes or chromosome segments.

In some embodiments utilizing a reference amount, the method includes (a) measuring the amount of genetic material on a chromosome or chromosome segment of interest; (b) comparing the amount from step (a) to a reference amount; and (c) identifying the presence or absence of a chromosomal abnormality in the genome of the fetus based on the comparison. In some embodiments, the reference amount is (i) a threshold value or (ii) an expected amount for a particular copy number hypothesis. In some embodiments, the reference amount is the amount determined for a chromosome from the same sample that is expected to be disomic. In some embodiments, the reference amount is the amount determined for the same chromosome from one or more different samples. In some embodiments, the reference amount is the mean or median of the values determined for two or more different chromosomes or different samples.

In some embodiments utilizing a reference chromosome, the method includes sequencing DNA from the blood sample (or fraction thereof) to obtain a plurality of sequence tags aligning to target loci. In some embodiments, the sequence tags are of sufficient length to be assigned to a specific target locus; the target loci are from a plurality of different chromosomes; and the plurality of different chromosomes comprise at least one first chromosome suspected of having an abnormal distribution in the sample and at least one second chromosome presumed to be normally distributed in the sample. In some embodiments, the plurality of sequence tags are assigned to their corresponding target loci. In some embodiments, the number of sequence tags aligning to the target loci of the first chromosome and the number of sequence tags aligning to the target loci of the second chromosome are determined. In some embodiments, these numbers are compared to determine the presence or absence of an abnormal distribution of the first chromosome.

In some embodiments, the fetal fraction is used in the fetal aneuploidy determination, such as to compare the observed difference between the amount of two chromosomes or chromosome segments to the difference that would be expected for a particular type of aneuploidy given the fetal fraction (see, e.g., US Publication No 2012/0190020; US Publication No 2012/0190021; US Publication No 2012/0190557; US Publication No 2012/0191358, which are each hereby incorporated by reference in its entirety). For example, the difference in the amount of chromosome 21 compared to a reference chromosome in a blood sample from a mother carrying a fetus with trisomy 21 increases as the fetal fraction increases. In some embodiments, the method includes amplifying two or more selected polymorphic nucleic acid regions from a first chromosome in the blood sample (or fraction thereof); amplifying two or more selected polymorphic nucleic acid regions from a second chromosome; and quantifying a relative frequency of each allele from the selected polymorphic nucleic acid regions to determine the fetal fraction in the sample; quantifying a relative frequency of the first and second chromosomes of interest (e.g., a relative frequency based on the selected polymorphic nucleic acid regions or based on selected non-polymorphic nucleic acid regions). In some embodiments, the method includes comparing the relative frequency of the first and second chromosomes of interest to the fetal fraction to determine the likelihood of a fetal aneuploidy. For example, the difference in amounts between the first and second chromosomes of interest can be compared to what would be expected given the fetal fraction for various possible aneuploidies (such as one or two extra copies of a chromosome of interest). In some embodiments, the method includes adjusting the relative frequency of the first and second chromosomes of interest based on the fetal fraction to determine the likelihood of a fetal aneuploidy. In some embodiments, the second chromosome of interest is expected or known to be disomic and is used as a reference chromosome.

### Methods for Screening for Disease-Linked Loci

In an embodiment, the prenatal testing includes testing for one or more single gene disorders. Single-gene disease diagnosis leverages the same targeted approach used for aneuploidy testing, and requires additional specific targets (see, e.g., U.S. application Ser. No. 13/300,235 (U.S. Publication No. 2012/0270212), U.S. application Ser. No. 13/683,604, and U.S. application Ser. No. 13/780,022, which are each hereby incorporated by reference in its entirety). In some embodiments, the allelic state is linked to a disease selected from the group consisting of cystic fibrosis, Huntington's disease, Fragile X, thallasemia, muscular dystrophy, Alzheimer, Fanconi Anemia, Gaucher Disease, Mucolipidosis IV, Niemann-Pick Disease, Tay-Sachs disease, Sickle cell anemia, Parkinson disease, Torsion Dystonia, and cancer.

In an embodiment, the single gene diagnosis is through linkage analysis. In some embodiments, the method involves phasing the abnormal allele with surrounding very tightly linked SNPs in the parents using information from first-degree relatives. Then PARENTAL SUPPORT™ may be run on the targeted sequencing data obtained from these SNPs to determine which homologs, normal or abnormal, were inherited by the fetus from both parents. As long as the SNPs are sufficiently linked, the inheritance of the genotype of the fetus can be determined very reliably. In some embodiments, the method comprises (a) including a set of SNP loci to densely flank a specified set of common disease; (b) reliably phasing the alleles from these added SNPs with the normal and abnormal alleles based on genetic data from various relatives; and (c) reconstructing the fetal haplotype, or set of phased SNP alleles on the inherited maternal and paternal homologs in the region surrounding the disease locus to determine the fetal genotype. In some embodiments, additional probes that are closely linked to a disease linked locus are added to the set of polymorphic loci being used for aneuploidy testing.

Reconstructing fetal diplotype is challenging because the sample is a mixture of maternal and fetal DNA. In some embodiments, the method incorporates relative information to phase the SNPs and disease alleles, then takes into account physical distance of the SNPs and recombination data from location specific recombination likelihoods and the data observed from the genetic measurements of the maternal plasma to obtain the most likely genotype of the fetus.

Phasing the diploid data from the parents can be performed as described herein or using known methods (see, e.g., PCT Publ. No. WO2009105531, filed Feb. 9, 2009; PCT Publ. No. WO2010017214, filed Aug. 4, 2009; and U.S. Utility application Ser. No. 13/683,604, filed Nov. 21, 2012, which are each hereby incorporated by reference in its entirety). In one embodiment, a parent can be phased by inference by measuring tissue from the parent that is haploid, for example by measuring one or more sperm or eggs. In one embodiment, the parent can be phased by inference using the measured genotypic data of a first degree relative such as the parent's parent(s) or siblings. In one embodiment, the parent can be phased by dilution where the DNA is diluted, in one or a plurality of wells, to the point where there is expected to be no more than approximately one copy of each haplotype in each well, and then measuring the DNA in the one or more wells. In one embodiment, the parent genotype can be phased by using computer programs that use population based haplotype frequencies to infer the most likely phase. In one embodiment, the parent can be phased if the phased haplotypic data is known for the other parent, along with the unphased genetic data of one or more genetic offspring of the parents. In some embodiments, the genetic offspring of the parents may be one or more embryos, fetuses, and/or born children. Some of these methods and other methods for phasing one or both parents are disclosed in greater detail in, e.g., U.S. Publ. No. 2011/0033862, filed Aug. 19, 2010; U.S. Publ. No. 2011/0178719, filed Feb. 3, 2011; U.S. Publ. No. 2007/0184467, filed Nov. 22, 2006; U.S. Publ. No. 2008/0243398, filed Mar. 17, 2008, which are each hereby incorporated by reference in its entirety.

### Paternity Testing

In an embodiment, the method is used for paternity testing (see, e.g, U.S. Publication No. 2012/0122701, filed Dec. 22, 2011, is which is hereby incorporated by reference in its entirety). In some embodiments, the present methods allow a plurality of polymorphic loci (such as SNPs) to be analyzed for use in the PARENTAL SUPPORT™ algorithm described herein to determine whether an alleged father in is the biological father of a fetus. For example, given the SNP-based genotypic information from the mother, and from a man who may or may not be the genetic father, and the measured genotypic information from the blood sample or a fraction thereof, it is possible to determine if the genotypic information of the male indeed represents that actual genetic father of the fetus. A simple way to do this is to simply look at the contexts where the mother is AA, and the possible father is AB or BB. In these cases, one may expect to see the father contribution half (AA|AB) or all (AA|BB) of the time, respectively. Taking into account the expected allele drop out (ADO), it is straightforward to determine whether or not the fetal SNPs that are observed are correlated with those of the possible father.

In some embodiments the method involves (i) obtaining genotypic measurements at a plurality of polymorphic loci on genetic material from the alleged father; (ii) obtaining genotypic measurements at a plurality of polymorphic loci on genetic material from the blood sample or a fraction thereof; (iii) determining on a computer the probability that the alleged father is the biological father of the fetus using the genotypic measurements; and (iv) establishing whether the alleged father is the biological father of the fetus using the determined probability that the alleged father is the biological father of the fetus. In various embodiments, the method also includes obtaining genotypic measurements at a plurality of polymorphic loci on genetic material from the mother, and the probability of each hypothesis is determined using the genotypic measurements made on the genetic material from the mother, the genetic material from the father, and the blood sample or a fraction thereof.

### Ex Vivo Methods

As described above, methods that can increase the fetal fraction tend to improve the accuracy of NIPT based on measuring fetal cfDNA in maternal blood.

In some embodiments of the invention, the method comprises amplifying the cfDNA. In some embodiments of the invention, the amplification consists of amplification multiple steps. In some embodiments of the invention, the amplification is a targeted PCR amplification, wherein the PCR primers target a set of loci. The goal of a targeted amplification is to preferentially amplify DNA from a specific set of loci, and not amplify any other DNA. However, in reality, even with carefully designed targeted PCR primers, other strands of DNA tend to amplify to varying degrees. An additional problem is that excess primers can result in a significant number of competing side reactions that both reduce the efficiency of the desired reaction and also result in a final reaction mixture with many other pieces of undesirable DNA. A method that is able to physically isolate the desired DNA segments would be of great benefit as it could increase the efficiency of a targeted PCR protocol significantly.

In some embodiments of the invention, one or a plurality of the PCR primers is covalently bonded to a matrix that can be physically separated from the reaction mixture. In some embodiments of the invention, one or a plurality of the PCR primers is covalently bonded to a magnetic particle and the magnet along with pendant DNA molecules could be physically separated from the reaction mixture using a magnetic field. In some embodiments of the invention, one or a plurality of the PCR primers is covalently bonded to a molecular moiety that can be used as a handle to physically affix the DNA, using non-covalent interactions, to a matrix that can be physically separated from the reaction mixture. In some embodiments of the invention, the handle could be biotin, and the moiety is streptavidin. In some embodiments, the non-covalent affixing could result from the tight interaction between biotin and streptavidin. In some embodiments of the invention, one or a plurality of the single stranded PCR primers is covalently bonded to a biotin molecule, and after amplification, the resultant double stranded DNA molecule can be physically isolated with a matrix that has streptavidin attached to it. In some embodiments of the invention, the handle could be a single stranded segment of DNA, and the non-covalent affixing could result from the tight interaction between the single stranded segment of DNA used as the handle and the complementary strand of DNA that could be affixed to a matrix. In some embodiments of the invention, one or a plurality of the single stranded PCR primers is covalently bonded to a biotin molecule, and after amplification, the resultant double stranded DNA molecule can be physically isolated with a matrix that has streptavidin attached to it. In some embodiments, the primer could be affixed to any pendant moiety that has a high affinity for another moiety that can be affixed to a matrix so as to provide a method for physical isolation of the targeted DNA.

In one embodiment, the method may comprise one or more of the following steps. Blood may be drawn from a pregnant woman. The blood sample may be centrifuged. The plasma fraction may be isolated. The plasma sample may be re-centrifuged and the plasma reisolated. The DNA may be isolated from the plasma. A “library” (meaning ligating Linkers, or Adaptors, to DNA fragments) may be made from the cfDNA. The ligation products with PCR primers, of which one is 5′ labeled with a Biotin, may be amplified. In some embodiments, the Forward primer may be Biotinylated. In some embodiments, the Reverse can be Biotinylated, with appropriate changes downstream. The amplified Library may be purified (e.g., by PCR column or Ampure beads).

The amplified products may be mixed with magnetic Streptavidin beads. The DNA with pendant Biotin may bind to the beads. The mixture may be made alkaline (e.g., with 0.1M NaOH), so that the non-biotinylated DNA strand denatures and can be washed away. A little TWEEN-20 (e.g., 0.025%) may be added to bead buffers and/or washes to prevent non-specific binding of DNA to the beads.

The beads may be incubated with PCR buffer (including polymerase) and Reverse Targeting primers (e.g., our 11,000-plex or 20,000-plex inner-R primers). If the Reverse Library amplification primer (above) had been Biotinylated, the Forward Targeting primers would be used. The mixture may be incubated without thermocycling such that the primers are extended by the polymerase on their targets. The beads may be washed again so that all non-used primers are removed and all extension products remain on the Biotinylated strands (i.e., washing with non-denaturing conditions).

The beads may be incubated with a PCR buffer (as above) with Forward Targeting primers (or Reverse Targeting, as above) for a number of cycles (e.g., about 5, about 10, about 15, about 20, or more than 20) to amplify the DNA. The amplified DNA may then be measured, for example, by sequencing according to standard sequencing protocols. The sequence measurements can be analyzed to determine the genetic status of the fetus.

In some embodiments, the “library” itself can be Biotinylated by having a Biotin on one of the Linker or Adaptor strands. With this approach, the Library amplification may be omitted. In some embodiments, the method comprises tailing plasma DNA with Biotin-nucleotides.

### Alternate Embodiments

Any of the embodiments disclosed herein may be implemented in digital electronic circuitry, integrated circuitry, specially designed ASICs (application-specific integrated circuits), computer hardware, firmware, software, or in combinations thereof. Apparatus of the presently disclosed embodiments can be implemented in a computer program product tangibly embodied in a machine-readable storage device for execution by a programmable processor; and method steps of the presently disclosed embodiments can be performed by a programmable processor executing a program of instructions to perform functions of the presently disclosed embodiments by operating on input data and generating output. The presently disclosed embodiments can be implemented advantageously in one or more computer programs that are executable and/or interpretable on a programmable system including at least one programmable processor, which may be special or general purpose, coupled to receive data and instructions from, and to transmit data and instructions to, a storage system, at least one input device, and at least one output device. Each computer program can be implemented in a high-level procedural or object-oriented programming language or in assembly or machine language if desired; and in any case, the language can be a compiled or interpreted language. A computer program may be deployed in any form, including as a stand-alone program, or as a module, component, subroutine, or other unit suitable for use in a computing environment. A computer program may be deployed to be executed or interpreted on one computer or on multiple computers at one site, or distributed across multiple sites and interconnected by a communication network.

Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Any of the methods described herein may include the output of data in a physical format, such as on a computer screen, or on a paper printout. In explanations of any embodiments elsewhere in this document, it should be understood that the described methods may be combined with the output of the actionable data in a format that can be acted upon by a physician. In addition, the described methods may be combined with the actual execution of a clinical decision that results in a clinical treatment, or the execution of a clinical decision to make no action. Some of the embodiments described in the document for determining genetic data pertaining to a target individual may be combined with the notification of a potential chromosomal abnormality, or lack thereof, with a medical professional, optionally combined with the decision to abort, or to not abort, a fetus in the context of prenatal diagnosis. Some of the embodiments described herein may be combined with the output of the actionable data, and the execution of a clinical decision that results in a clinical treatment, or the execution of a clinical decision to make no action.

In some embodiments, a method is disclosed herein for determining the ploidy status of a chromosome in a gestating fetus, or the paternity status of the gestating fetus in relation to an alleged father. In some embodiments, a method is disclosed herein for generating a report disclosing the determined ploidy status or allelic status (e.g., inheritance of a disease-linked locus) of a chromosome in a gestating fetus, or the paternity status of the gestating fetus. In some embodiments, the method comprises one or more of the following steps: a woman consuming a beverage or food that contains sugar or other stimulating substance, causing flexion or compression around the abdominal region of a woman, obtaining a first sample that contains DNA from the mother of the fetus and DNA from the fetus; obtaining genotypic data from one or both parents of the fetus; preparing the first sample by isolating the DNA so as to obtain a prepared sample; amplifying the DNA in the prepared sample using PCR; amplifying the DNA in the prepared sample using target PCR; measuring the DNA in the prepared sample at a plurality of polymorphic loci; measuring the DNA in the prepared sample using massively parallel shotgun sequencing; calculating, on a computer, allele counts or allele count probabilities at the plurality of polymorphic loci from the DNA measurements made on the prepared sample; calculating the number of sequence reads that originate from a target chromosome or chromosomal region and comparing that number to a number of sequence reads that originate from a control chromosome(s) or chromosomal regions(s); calculating a z-score based on the relative number of reads that originate from the target chromosome; creating, on a computer, a plurality of ploidy hypotheses concerning expected allele count probabilities at the plurality of polymorphic loci on the chromosome for different possible ploidy states of the chromosome; building, on a computer, a joint distribution model for allele count probability of each polymorphic locus on the chromosome for each ploidy hypothesis using genotypic data from the one or both parents of the fetus; determining, on a computer, a relative probability of each of the ploidy hypotheses using the joint distribution model and the allele count probabilities calculated for the prepared sample; calling the ploidy state of the fetus by selecting the ploidy state corresponding to the hypothesis with the greatest probability; and generating a report disclosing the determined ploidy status.

In another embodiment, a pregnant woman wants to know whether the gestating fetus has Down syndrome, Turner Syndrome, Prader Willi syndrome, or some other whole chromosomal abnormality. The obstetrician instructs the woman to drink a sugary beverage, and after waiting for a period of time, takes a blood draw from the mother. The blood is sent to a laboratory, where a technician centrifuges the maternal sample to isolate the plasma. Massively parallel shotgun sequencing is performed on the plasma sample. The sequencing transforms the information that is encoded molecularly in the DNA into information that is encoded electronically in computer hardware. The number of sequence reads that map to the chromosomes of interest are normalized and a z-score is calculated. From the z-score, it is determined that the fetus has Down syndrome. A report is printed out, or sent electronically to the pregnant woman's obstetrician, who transmits the diagnosis to the woman. The woman, her husband, and the doctor sit down and discuss their options. The couple decides to terminate the pregnancy based on the knowledge that the fetus is afflicted with a trisomic condition.

In an embodiment, a pregnant women may want to know if her gestating fetus is afflicted with a chromosomal abnormality, and also if her husband is the genetic father. The mother may be instructed to drink a sugary beverage, waits in her doctor's office for short period of time (for example, between one and one hundred minutes). A phlebotomist may then draws a sample of blood from both the mother and father. A clinician may isolate the plasma from the maternal blood, and purify the DNA from the plasma. A clinician may also isolate the buffy coat layer from the maternal blood, and prepare the DNA from the buffy coat. A clinician may also prepare the DNA from the paternal blood sample. The clinician may use molecular biology techniques to append universal amplification tags to the DNA in the DNA derived from the plasma sample. The clinician may amplify the universally tagged DNA. The clinician may preferentially enrich the DNA by a number of techniques including capture by hybridization with hybrid capture probes and targeted PCR (see, for example, U.S. application Ser. No. 13/683,604, filed Nov. 21, 2012, which is hereby incorporated by reference in its entirety for the teachings therein, such as those related to enrichment and/or amplification methods). The targeted PCR may involve nesting, hemi-nesting or semi-nesting, or any other approach to result in efficient enrichment of the plasma derived DNA. The targeted PCR may be massively multiplexed, for example with as many as about 2,000 or even about 20,000 primers in one reaction, where the primers target SNPs on one or a plurality of chromosomes, for example chromosomes 13, 18, 21, X and Y. The selective enrichment and/or amplification may involve tagging each individual molecule with different tags, molecular barcodes, tags for amplification, and/or tags for sequencing. The clinician may then sequence the plasma sample, and also possibly also the prepared maternal and/or paternal DNA. The molecular biology steps may be executed either wholly or partly by a diagnostic box. The sequence data may be fed into a single computer or to another type of computing platform such as may be found in ‘the cloud’. The computing platform may calculate allele counts at the targeted polymorphic loci from the measurements made by the sequencer. The computing platform may create a plurality of ploidy hypotheses pertaining to nullsomy, monosomy, disomy, matched trisomy, and unmatched trisomy for each of chromosomes 13, 18, 21, X and Y. The computing platform may build a joint distribution model for the expected allele counts at the targeted loci on the chromosome for each ploidy hypothesis for each of the five chromosomes being interrogated. The computing platform may determine a probability that each of the ploidy hypotheses is true using the joint distribution model and the allele counts measured on the preferentially enriched DNA derived from the plasma sample. The computing platform may call the ploidy state of the fetus, for each of chromosome 13, 18, 21, X and Y by selecting the ploidy state corresponding to the germane hypothesis with the greatest probability. A report may be generated comprising the called ploidy states, and it may be sent to the obstetrician electronically, displayed on an output device, or a printed hard copy of the report may be delivered to the obstetrician. The obstetrician may inform the patient and optionally the father of the fetus, and they may decide which clinical options are open to them, and which is most desirable.

Any of the embodiments described in this document could be used in combination with any other method embodiments described in this or other related applications that related to non-invasive prenatal testing.

### EXPERIMENTAL SECTION

The presently disclosed embodiments are described in the following Example, which is set forth to aid in the understanding of the disclosure, and should not be construed to limit in any way the scope of the disclosure as defined in the claims which follow thereafter. The following example is put forth so as to provide those of ordinary skill in the art with a complete disclosure and description of how to use the described embodiments, and is not intended to limit the scope of the disclosure nor is it intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by volume, and temperature is in degrees Centigrade. It should be understood that variations in the methods as described may be made without changing the fundamental aspects that the experiments are meant to illustrate.

### Example 1

A total of 40 mL of blood were collected from each subject into two to four CELL-FREE™ DNA tubes (STRECK); 20 mL were collected before drinking the orange juice (pre-OJ), and 20 mL were collected after drinking the orange juice (post-OJ) and waiting 20 minutes. The pre-OJ and post-OJ samples were treated as separate samples. Plasma was isolated from each sample via a double centrifugation protocol of 2000 g for 20 min, followed by 3220 g for 30 min, with supernatant transfer following the first spin. cfDNA was isolated from 7-20 mL plasma using the QIAGEN QIAamp Circulating Nucleic Acid kit and eluted in 45 uL TE buffer. Pure maternal genomic DNA was isolated from the buffy coat obtained following the first centrifugation, and pure paternal genomic DNA was prepared similarly from a blood, saliva or buccal sample.

Samples were pre-amplified for 15 cycles using 11,000 target-specific assays and an aliquot was transferred to a second PCR reaction of 15 cycles using nested primers. Finally, samples were prepared for sequencing by adding barcoded tags in a third 12-cycle round of PCR. Thus, 11,000 targets were amplified in a single reaction; the targets included SNPs found on chromosomes 13, 18, 21, X, and Y. The amplicons were then sequenced using an ILLUMINA GAIIx or HiSEQ sequencer. The sequencing data was then analyzed using the PARENTAL SUPPORT™ methodology.

All patents, patent applications, and published references cited herein are hereby incorporated by reference in their entirety. While the methods of the present disclosure have been described in connection with the specific embodiments thereof, it will be understood that it is capable of further modification. Furthermore, this application is intended to cover any variations, uses, or adaptations of the methods of the present disclosure, including such departures from the present disclosure as come within known or customary practice in the art to which the methods of the present disclosure pertain, and as fall within the scope of the appended claims.

What is claimed is:

1. A method for performing non-invasive prenatal testing on a pregnant woman, the method comprising in sequential order:
(a) administering (i) a nutritious composition or (ii) a stimulant to a pregnant woman in an amount sufficient to increase the fetal fraction in the blood, plasma, or serum of the pregnant woman;
(b) obtaining a blood sample from the pregnant woman; and
(c) performing non-invasive prenatal testing on the blood sample or a fraction thereof.

2. The method of claim 1, wherein the administration comprises having the woman consume a food or drink.

3. The method of claim 1, wherein the composition comprises at least 20 g carbohydrate.

4. The method of claim 3, wherein the composition comprises at least 40 g carbohydrate.

5. The method of claim 1, wherein the composition comprises at least 0.3 g carbohydrate per kg of body weight.

6. The method of claim 5, wherein the composition comprises at least 0.6 g carbohydrate per kg of body weight.

7. The method of claim 1, wherein the composition comprises fructose.

8. The method of claim 1, wherein the composition comprises at least 100 calories.

9. The method of claim 8, wherein the composition comprises at least 190 calories.

10. The method of claim 1, wherein the stimulant comprises caffeine.

11. The method of claim 10, wherein the stimulant comprises at least 40 mg caffeine.

12. The method of claim 1, wherein step (a) increases the fetal fraction in the blood, plasma, or serum of the pregnant woman by at least 10%.

13. The method of claim 12, wherein step (a) increases the fetal fraction in the blood, plasma, or serum of the pregnant woman by at least 20%.

14. The method of claim 1, wherein the time between step (a) and step (b) is between 1 and 100 minutes.

15. The method of claim 1, wherein the time between step (a) and step (b) is less than 30 minutes.

16. The method of claim 1, wherein the prenatal testing determines the presence or absence of a chromosomal abnormality in the genome of the fetus.

17. The method of claim 16, wherein the chromosomal abnormality is selected from the group consisting of monosomy, uniparental disomy, trisomy, mosaicism, other aneuploidies, unbalanced translocations, insertions, deletions, and combinations thereof.

18. The method of claim 16, wherein the prenatal testing comprises determining whether the individual has Down syndrome, Edwards syndrome, Patau syndrome, Klinefelters syndrome, 47,XXX, 47,XYY, Turner syndrome, triploidy, DiGeorge syndrome, Cri du Chat syndrome, Angelman syndrome, Praeder-Willi syndrome, Wolf-Hirschhorn syndrome, Smith-Magenis syndrome, Williams-Beuren syndrome, Phelan-McDermid syndrome, or Sotos Syndrome.

19. The method of claim 1, wherein the prenatal testing determines the presence or absence of a disease-linked locus in the genome of the fetus.

20. The method of claim 19, wherein the locus is linked to a disease selected from the group consisting of cystic fibrosis, Huntington's disease, Fragile X, thallasemia, muscular dystrophy, Alzheimer, Fanconi Anemia, Gaucher Disease, Mucolipidosis IV, Niemann-Pick Disease, Tay-Sachs disease, Sickle cell anemia, Parkinson disease, Torsion Dystonia, and cancer.

21. The method of claim 1, wherein the prenatal testing determines whether or not an alleged father is the biological father of the fetus.

22. The method of claim 16, wherein the prenatal testing comprises
(a) measuring the amount of genetic material on a chromosome or chromosome segment of interest;
(b) comparing the amount from step (a) to a reference amount; and
(c) identifying the presence or absence of a chromosomal abnormality in the genome of the fetus based on the comparison.

23. The method of claim 16, wherein the prenatal testing comprises
(a) sequencing DNA from in the blood sample or fraction thereof to obtain a plurality of sequence tags aligning to target loci; wherein the sequence tags are of sufficient length to be assigned to a specific target locus; wherein the target loci are from a plurality of different chromosomes; and wherein the plurality of different chromosomes comprise at least one first chromosome suspected of having an abnormal distribution in the sample and at least one second chromosome presumed to be normally distributed in the sample;
(b) assigning on a computer the plurality of sequence tags to their corresponding target loci;
(c) determining on a computer a number of sequence tags aligning to the target loci of the first chromosome and a number of sequence tags aligning to the target loci of the second chromosome; and
(d) comparing on a computer the numbers from step (c) to determine the presence or absence of an abnormal distribution of the first chromosome.

24. The method of claim 16, wherein the prenatal testing comprises
making genotypic measurements at a plurality of polymorphic loci in the blood sample or fraction thereof;
determining, on a computer, a fetal fraction in the blood sample or fraction thereof given the genotypic measurements of the blood sample or fraction thereof;
creating, on a computer, a set of ploidy state hypothesis for a chromosome or chromosome segment of interest in the fetus;
determining, on the computer, the probability of each of the hypotheses given the genetic measurements of the blood sample or fraction thereof and the fetal fraction; and
using the determined probabilities of each hypothesis to determine the most likely copy number of the chromosome or chromosome segment of interest in the genome of the fetus.

25. The method of claim 16, wherein the prenatal testing comprises
amplifying two or more selected polymorphic nucleic acid regions from a first chromosome in the blood sample or fraction thereof;
amplifying two or more selected polymorphic nucleic acid regions from a second chromosome;
quantifying a relative frequency of each allele from the selected polymorphic nucleic acid regions to determine the fetal fraction in the sample;
quantifying a relative frequency of the first and second chromosomes of interest; and
comparing the relative frequency of the first and second chromosomes of interest to the fetal fraction to determine the likelihood of a fetal aneuploidy.

26. The method of claim 1, wherein the prenatal testing is performed by measuring the cell free DNA found in the maternal plasma.

27. The method of claim 26, wherein the cell free DNA is measured by sequencing.

28. The method of claim 26, wherein the cell free DNA is amplified prior to measurement.

29. A report comprising a result from the non-invasive prenatal testing method of claim 1.

30. A method increasing the fetal fraction in the blood of a pregnant woman, the method comprising in sequential order:
(a) administering (i) a nutritious composition or (ii) a stimulant to a pregnant woman in an amount sufficient to increase the increase the fetal fraction in the blood, plasma, or serum of the pregnant woman;
(b) obtaining a blood sample from the pregnant woman; and
(c) measuring the fetal fraction in the blood sample or a fraction thereof.
